(19) United States
(12) Patent Application Publication
(10) Pub. No.: US 2016/0263362 A1
(43) Pub. Date: Sep. 15, 2016

(54) MICRONEEDLE ARRAYS FOR ACTIVE AGENT DELIVERY

(71) Applicant: Transderm, Inc., Salt Lake City, UT (US)

(72) Inventors: Roger L. Kaspar, Santa Cruz, CA (US); Tycho Speaker, Santa Cruz, CA (US)

(73) Assignee: TRANSDERM, INC., Salt Lake City, UT (US)

(21) Appl. No.: 14/788,439

(22) Filed: Jun. 30, 2015

Related U.S. Application Data
(63) Continuation of application No. 13/157,198, filed on Jun. 9, 2011, now abandoned.
Application No. 13/157,198 is a continuation-in-part of application No. 12/187,268, filed on Aug. 6, 2008, now Pat. No. 8,366,677.
(60) Provisional application No. 61/353,108, filed on Jun. 9, 2010.
(60) Provisional application No. 60/963,725, filed on Aug. 6, 2007.
(60) Provisional application No. 60/994,568, filed on Sep. 19, 2007.

(51) Int. Cl. A61M 37/00; A61K 9/00; C12N 15/113; A61K 31/713; A61K 39/00; A61B 17/20; A61K 38/21

(52) U.S. Cl. CPC A61M 37/0015; A61B 17/205; A61K 9/0021; A61K 38/21; A61K 31/713; A61K 39/00; C12N 15/113; A61M 2037/0023; A61M 2037/0061; A61M 2202/30; A61M 2037/0046; A61M 2205/583; A61K 2039/54; C12N 2310/14

(57) ABSTRACT

The present invention provides for microneedle arrays and related systems and methods. Particularly, microneedle arrays that are configured to deliver active agents, including nucleic acids and vaccines, are provided. Additional related methods of vaccinating and minimizing the amount of vaccine necessary for effective inoculation are also provided.

### PRIORITY DATA

This application is a continuation of U.S. patent application Ser. No. 13/157,198, filed Jun. 9, 2011, which claims the benefit of U.S. Provisional Patent Application Ser. No. 61/353,108, filed on Jun. 9, 2010, and which is a continuation-in-part of U.S. patent application Ser. No. 12/187,268, filed on Aug. 6, 2008, which claims the benefit of U.S. Provisional Application Ser. Nos. 60/963,725, filed Aug. 6, 2007, and 60/994,568, filed Sep. 19, 2007, all of which are incorporated herein by reference.

### BACKGROUND

Transdermal delivery of various active agents has been a common and effective way for delivering active agents to subjects. Unfortunately, there are significant challenges associated with the delivery of some types of active agents due to their complexity and size. Examples of active agents that can be difficult to deliver transdermally include nucleic acids, vaccines, proteins, microorganisms, and the like. A major obstacle for the delivery of these active agents is overcoming the stratum corneum barrier. The natural function of the stratum corneum is to prevent water loss and exclude external agents from entering the body with a molecular cut off of about ˜500 Da. With this in mind, research continues into methods for providing effective transdermal delivery of these types of active agents.

### SUMMARY OF THE INVENTION

The present invention provides microneedle arrays, associated systems, and methods that can be used to deliver various therapeutic agents, including nucleic acids and vaccines. In one embodiment, a method of delivering a therapeutically effective amount of an active agent to a subject is provided. A microneedle array comprising a base portion and a plurality of microneedles attached to the base portion is provided. The microneedles can have a therapeutically effective amount of an active agent included therein. The microneedles are applied a skin surface of a subject in a manner sufficient to embed the microneedles into the skin surface. The base of the microneedle array can then be separated from the microneedles such that the microneedles remain embedded in the skin surface and the base is removed from the skin surface. The microneedles can be maintained in the skin surface until the microneedles are absorbed (i.e. dissolved in the skin), or otherwise removed by the subject.

In another embodiment, a method of providing visual verification of microneedle placement in a skin surface is provided. The method includes providing a microneedle array including a plurality of microneedles attached to the microneedle array, wherein at least one of the microneedles includes an indicator. The microneedle array is applied a skin surface of a subject such that the microneedles are embedded into the skin surface and then verifying of successful application of the microneedles can be made through observation of the indicators presence in the skin.

In another embodiment, a method of vaccinating is provided. The method includes provoking an immune response, including a localized immune response, in a subject in need of vaccination and delivering a vaccine concomitantly with the provocation of the immune response. In yet another embodiment, such a method may include providing a microneedle array comprising a base and microneedle attached to the base. The microneedles can be loaded with an amount of a vaccine sufficient to provide inoculation to the subject. The microneedle array can be applied to a skin surface of a subject such that the microneedles are embedded into the skin surface. After application, the base of the microneedle array can be separated from the microneedles such that the microneedles remain embedded in the skin surface and the base is removed from the skin surface. The microneedles can be maintained in the skin surface until the microneedles are absorbed by the subject.

In an additional embodiment, a method of minimizing the amount of vaccine required to effectively vaccinate a subject against a disease for which the vaccine is effective is provided. The method includes stimulating an immune response in the subject at a vaccination site while concomitantly delivering of the vaccination.

A microneedle device is also provided. In one aspect, the microneedle device includes a therapeutically effective amount of an active agent, a base, and a plurality of microneedles attached to the base. The microneedles can have at least one external longitudinal channel that contains at least a portion of the active agent. In another embodiment, a microneedle device is provided that includes a therapeutically effective amount of an active agent, a base, and a plurality of microneedles attached to the base. The microneedles can include a lower portion and an upper portion. The lower portion can be adjacent to the base and the upper portion can be opposite the base. The lower portion and said upper portion can be compositionally distinct.

In another embodiment, a system for delivering siRNA to a subject is provided. The system can include a therapeutically effective amount of a self-delivering siRNA and a microneedle array. The microneedle array can include a base and a plurality of microneedles attached to the base. The microneedles can include a bioabsorbable/biodegradable material and can be configured to be detached from the base after being embedded in a skin surface.

### BRIEF DESCRIPTION OF THE DRAWINGS

Additional features and advantages of the invention will be apparent from the detailed description which follows, taken in conjunction with the accompanying drawings, which together illustrate, by way of example, features of the invention; and, wherein:

FIGS. 1A-1F shows a schematic of one embodiment of a method of fabrication of a microneedle array and example images. 1A. Pin template (top, left panel) and glass slide (bottom, left panel) are covered with a thin film of viscous (20%) polyvinyl alcohol (PVA) solution (blue). The pin template is placed in contact with the PVA solution (middle panel). Microneedles are produced by withdrawing the pins as the film is drying, forming fiber-like structures (Right panel). 1B. Enlarged view of fibers. 1C. Protrusions are subsequently trimmed to the desired length and tip shape. 1D. Microneedle array supported by a glass substrate, with a penny to show scale. 1E. Micrograph of one microneedle after trimming to 1 mm length, showing beveled structure that facilitates skin penetration, with a human hair (˜100 μm diameter) to show scale. 1F. Microneedle arrays loaded alternatively with fluorescein (green) or R-phycoerythrin (red).

FIGS. 2A-2B shows imaging of individual microneedle penetration sites in vivo and microneedle plug visualization in skin sections. 2A. Microneedle arrays were loaded with siGLO Red (a fluorescently-tagged siRNA mimic, ˜20 ng/microneedle) and applied to the left footpad. As a control, 0.5 μg of siGLO Red (in 50 μl PBS) was injected intradermally into the right footpad. Mice were immediately intravitally imaged for fluorescence using the Xenogen IVIS 200 system. Localized fluorescence corresponding to individual microneedle penetration sites was observed following microneedle array application. 2B. Fluorescence microscopy of 10 μm skin sections showing a microneedle depot loaded with siGLO Red (longer exposure time [not shown] shows initial release of siGLO Red) demonstrating drug release to the epidermis. Sections were stained with DAPI to visualize nuclei (bar=10 μm).

FIGS. 3A-3D shows fluorescence microscopy analysis of Accell Red siRNA distribution in mouse footpad skin. Transgenic CBL/hMGFP mouse footpads were treated with two (3×5) microneedle arrays loaded with Accell Red (DY-547-labelled) non-targeting siRNA. Mice were sacrificed at 1.5 (3A, 3B) and 6 (3C, 3D) h after microneedle array application and footpad skin sections removed for analysis. Accell Red siRNA (red fluorescence, middle panels) distributes through dermis (d) and epidermis (ep). With this needle design and length, the red fluorescence signal is detected in the basal (b) and spinosum (s) layers at 1.5 h (3A, 3B) and reaches the granular layer (g) and stratum corneum (sc) at 6 h. Sections were stained with DAPI to visualize nuclei (right panel). Brightfield fluorescence overlay (left panel). Bar=20 μm.

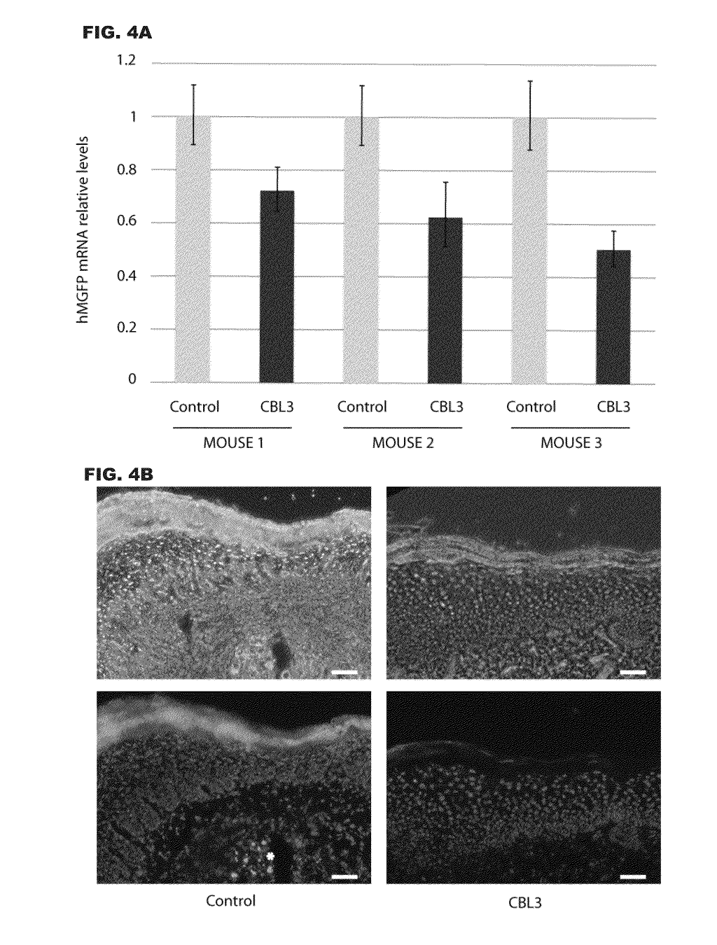

FIGS. 4A-4D shows CBL3 Accell siRNA-loaded microneedle arrays inhibit hMGFP expression in mouse footpad skin. Transgenic CBL/hMGFP mouse footpads were treated every two days with three (3×5) microneedle arrays loaded with either CBL3 or non-specific control Accell siRNA for 12 days. 4A. RT-qPCR analysis of Tg CBL/hMGFP mice treated with CBL3 Accell siRNA. Total RNA, isolated from paw palm skin of three mice treated with CBL3 Accell siRNA (right footpad) or a non-specific control Accell siRNA (left footpad), was reverse transcribed and hMGFP mRNA levels quantified by qPCR. The hMGFP levels were normalized to K14 levels (endogenous control). Each bar corresponds to the mean of three replicates. Bars indicate standard error. 4B. Fluorescence microscopy of frozen skin sections prepared from treated mice. Mice were sacrificed and frozen skin sections (10 μm) prepared. hMGFP expression (or lack thereof) was visualized by fluorescence microscopy of samples from mouse footpads treated with non-specific Accell siRNA (left panel) or CBL3 (right panel) Accell siRNA. Upper panel shows brightfield overlay and bottom panel shows fluorescence only. Scale bar is 50 μm. Nuclei are visualized by DAPI stain (blue). 4C. In vivo quantification of hMGFP fluorescence. Three mice were treated with CBL3 Accell siRNA (right paw) and non-specific Accell siRNA (left paw) and imaged with the CRi Maestro imaging system during treatment. Quantification of the region of interest adjusted to the palm of each mouse was performed using Maestro quantification software after background subtraction. The ratio in the average signal (counts/s/mm²) of CBL3 Accell treated palm and non-specific Accell treated palm normalized to day 0 is reported in the graph. 4D. hMGFP expression in mouse 3 imaged during treatment with the CRi Maestro imaging system. The right paw was treated with CBL3 Accell siRNA while the left paw was treated with non-specific control Accell siRNA. Images were taken using auto-expose settings and un-mixed using previously defined spectra and autofluorescence. The images are pseudocolored green.

FIGS. 5A-5B shows analysis of fLuc reporter gene expression in mouse ear and footpads following microneedle array-mediated delivery of expression plasmids. 5A. Ear delivery. The ear on the right was treated with a microneedle array loaded with approximately 12 ng/microneedle of pGL3-CMV-Luc plasmid (12 microneedles). The ear on the left was treated with the delivery device loaded with PBS vehicle alone. Microneedle arrays were inserted into the ear for 20 min. After 24 h, luciferase expression was determined following IP luciferin injection by whole animal imaging using the Xenogen IVIS 200 in vivo system (red is the highest expression level, blue lowest). 5B. Footpad delivery. Right footpads were treated with microneedle arrays (12 microneedles) loaded with luciferase expression plasmid for two consecutive days and analyzed as described above. Left footpads were treated with microneedle arrays loaded with PBS vehicle alone (control).

FIGS. 6A-6F shows fluorescence microscopy of mouse footpad skin sections demonstrates microarray-mediated siGLO Red (fluorescently-labeled siRNA mimic) delivery to the epidermis (or dermis). When needles were detected in the epidermis, fluorescent signal was found laterally dispersed from the delivery site (6A, 6B; ˜90 min timepoint) and progressively decreased through lower epidermal layers and dermis (bar=20 μm). In other occasions (6C, 6D), diffusion was detected in both the dermis and epidermis (bar=10 μm). When needles were detected in the dermis the fluorescent signal was rapidly dispersed (6E, 6F; this image was taken 30 min after application, bar=50 μm). Sections were stained with DAPI to visualize nuclei.

Reference will now be made to the exemplary embodiments illustrated, and specific language will be used herein to describe the same. It will nevertheless be understood that no limitation of the scope of the invention is thereby intended.

### DETAILED DESCRIPTION OF EXAMPLE EMBODIMENT(S)

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting.

In describing and claiming the present invention, the following terminology will be used.

The singular forms “a,” “an,” and “the” include plural referents unless the context clearly dictates otherwise. Thus, for example, reference to “a microneedle” includes reference to one or more microneedles, and reference to “the polymer” includes reference to one or more materials.

As used herein, the term “about” is used to provide flexibility to a numerical range endpoint by providing that a given value may be “a little above” or “a little below” the endpoint.

The term “subject” refers to a mammal that may benefit from the administration using a transdermal device or method of this invention. Examples of subjects include humans, and other animals such as horses, pigs, cattle, dogs, cats, rabbits, and aquatic mammals.

As used herein, the term “active agent” or “drug” are used interchangeably and refer to a pharmacologically active substance or composition.

As used herein, the term “concomitantly” when used to describe the stimulation of an immune response in conjunction with the delivery of a vaccination refers to the overlap in time between the stimulation of the immune response and the vaccination. The actual stimulation of the immune system need not take place simultaneously with the actual administration of the vaccination. Rather, concomitant stimulation of the immune system with delivery of the vaccine merely requires that the immune system be stimulated to an elevated level shortly before, during, or shortly after administration or delivery of the vaccine. In short, any combination or sequence of administration and immunostimulation which results in improved recognition by the body of the vaccine is suitably qualified as concomitant administration. Generally speaking, when stimulation occurs before vaccination, the stimulation should be within a time period that the immune system is still at an elevated level when the vaccine is delivered/administered. In some cases, when the stimulation occurs after vaccination, the stimulation should be within a time period that the immune system is stimulated to the elevated level within 24 hours of delivery of the vaccination, preferably within 12 hours of vaccination.

As used herein, “self-delivery siRNA” or “self-delivering siRNA” can be used interchangeably and refer to RNAi compounds that are modified to enable delivery to target cells and organs and efficient cellular uptake without the use of a delivery vehicle such as a transfection reagent. Non-limiting examples of commercially available self-delivering siRNA compounds that can be used in the systems and methods of the present invention include rxRNA™ compounds such as rxRNAori™, rxRNAsolo™ and sd-rxRNA™ by RXI Pharmaceuticals Corporation and Accell® siRNA by Thermo Scientific Dharmacon®.

As used herein, an “external longitudinal channel” is defined as being a channel or groove that runs along at least a portion of the longitudinal axis of the microneedle and which is open to the outside or exterior of the microneedle along at least a portion of its length.

As used herein, the term “substantially” refers to the complete or nearly complete extent or degree of an action, characteristic, property, state, structure, item, or result. As used herein, sequences, compounds, formulations, delivery mechanisms, or other items may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.5 to 10 g” should be interpreted to include not only the explicitly recited values of about 0.5 g to about 10.0 g, but also include individual values and sub-ranges within the indicated range. Thus, included in this numerical range are individual values such as 2, 5, and 7, and sub-ranges such as from 2 to 8, 4 to 6, etc. This same principle applies to ranges reciting only one numerical value. Furthermore, such an interpretation should apply regardless of the breadth of the range or the characteristics being described.

Unless otherwise defined, all technical and scientific terms used herein have the same meaning as commonly understood to one of ordinary skill in the art to which this invention belongs. Although any methods, devices and materials similar or equivalent to those described herein can be used in the practice or testing of the invention, representative methods, devices, and materials are described below.

It is noted that when discussing the devices, systems and associated methods, each of these discussions can be considered applicable to each of these embodiments, whether or not they are explicitly discussed in the context of that embodiment. Thus, for example, in discussing a biodegradable polymer that can be used in the methods of vaccinating, such a polymer can also be used for a microneedle array, and vice versa.

Accordingly, the present invention provides microneedle arrays, associated systems, and methods that can be used to deliver various therapeutic agents, including nucleic acids and vaccines. In one embodiment, a method of delivering a therapeutically effective amount of an active agent to a subject is provided. A microneedle array comprising a base portion and a plurality of microneedles attached to the base portion are provided. The microneedles can have a therapeutically effective amount of an active agent included therein. The microneedles are applied to a skin surface of a subject in a manner sufficient to embed the microneedles into the skin surface. The base of the microneedle array can then be separated from the microneedles such that the microneedles remain embedded in the skin surface and the base is removed from the skin surface. The microneedles can be maintained in the skin surface until the microneedles are absorbed by the subject.

In another embodiment, a method of providing visual verification of microneedle placement in a skin surface is provided. The method includes providing a microneedle array including a plurality of microneedles attached to the microneedle array, wherein at least one of the microneedles includes an indicator. The microneedle array is applied a skin surface of a subject such that the microneedles are embedded into the skin surface and then verifying successful application of the microneedles can be made through observation of the indicators presence in the skin.

In another embodiment, a method of vaccinating is provided. The method includes provoking an immune response in a subject in need of vaccination and delivering a vaccine concomitantly with the provocation of the immune response. In yet another embodiment, a method of vaccinating a subject in thereof is provided. The method includes providing a microneedle array comprising a base and microneedle attached to the base. The microneedles can be loaded with an amount of a vaccine sufficient to provide inoculation to the subject. The microneedle array can be applied to a skin surface of a subject such that the microneedles are embedded into the skin surface. After application, the base of the microneedle array can be separated from the microneedles such that the microneedles remain embedded in the skin surface and the base is removed from the skin surface. The microneedles can be maintained in the skin surface until the microneedles are absorbed by the subject.

In an additional embodiment, a method of minimizing the amount of vaccine required to effectively vaccinate a subject against a disease for which the vaccine is effective is provided. The method includes stimulating an immune response in the subject at a vaccination site while concomitantly with delivery of the vaccination.

A microneedle device is also provided. The microneedle device includes a therapeutically effective amount of an active agent, a base, and a plurality of microneedles attached to the base. The microneedles can have at least one external longitudinal channel that contains at least a portion of the active agent. In another embodiment, a microneedle device is provided that includes a therapeutically effective amount of an active agent, a base, and a plurality of microneedles attached to the base. The microneedles can include a lower portion and an upper portion. The lower portion can be adjacent to the base and the upper portion can be opposite the base. The lower portion and said upper portion can be compositionally distinct.

In another embodiment, a system for delivering siRNA to a subject is provided. The system can include a therapeutically effective amount of a self-delivering siRNA and a microneedle array. The microneedle array can include a base and a plurality of microneedles attached to the base. The microneedles can include a bioabsorbable/biodegradable material and can be configured to be detached from the base after being embedded in a skin surface.

A major obstacle to transdermal delivery of certain active agents, including siRNAs and vaccines, is the challenge of getting the active agents through the stratum corneum barrier. The natural function of the stratum corneum is to prevent water loss and exclude external agents from entering the body with a molecular cut off of about ˜500 Da. The microneedles array of the present invention can effectively circumvent this barrier by direct skin penetration. Further, the microneedles arrays of the present invention can be made of bioabsorbable/biodegradable materials and provide the added benefit that the tips may be left in the skin and can act as reservoirs, releasing the active agents over extended periods of time while the microneedles are dissolved and absorbed into the body of the subject

The microneedle arrays of the present invention can be configured to include a base portion and a plurality of microneedles attached to the base. The microneedles can be substantially perpendicular to the base and can be configured to be detached from the based after they are embedded into a skin surface. Once the microneedles are embedded into the skin surface, the base can be separated from the microneedles and removed from the skin surface. By leaving the microneedles in the skin surface, the active agents present in the microneedles can continue to be delivered.

The microneedles of the present invention can have at least one external longitudinal channel which is open along at least a portion of its length to the outside of the microneedle. In some embodiments, the microneedles can have several of these longitudinal channels. Such longitudinal channels are distinct from enclosed tubes that can be present in the microneedles and can have openings at the tops (tip end) or bottoms (base end) but not along the length of the microneedles. The longitudinal channels can be loaded with the active agents delivered by the microneedle arrays. The longitudinal channels can act to increase the surface area of the microneedle thereby increasing the rate of release of the active agent into the subject. Further, the longitudinal channels can also act to facilitate the loading of the microneedles with the active agent or other compounds.

The microneedles can also be configured to have a lower portion and an upper portion, the lower portion being adjacent to the base and the upper portion being opposite the base or at the tip of the microneedle. In one embodiment, the lower portion and the upper portion of the microneedle can be compositionally distinct. In one embodiment, the active agent can be present only in the upper portion of the microneedle. In another embodiment, the active agent can be present only in the lower portion of the microneedle. In yet a further embodiment, the microneedle can include two different active agents, a first active agent being present in the lower portion and a second active agent being present in the upper portion.

The microneedles of the microneedle arrays of the present invention can be made of bioabsorbable/biodegradable materials. Non-limiting examples of bioabsorbable/biodegradable materials that can be used include polyvinyl alcohol, polyvinylpyrrolidone, and other polymers, chitin, carboxymethyl cellulose, methyl cellulose, ethyl cellulose, sodium alginate, carrageenan, carbomer, alginates, and other polysaccharides, shellac, zein and other proteins, glucose, sucrose, maltose, trehalose, amylose, dextrose, fructose, mannose, galactose, fructose, and other sugars, mixtures thereof, and copolymers thereof, generally only limited by the ability to create a viscous solution in a solvent that can volatilize during formation of the fiber-like needle structure. In the present invention, polymeric or glassy materials that are water-soluble are preferred due to their ability to hydrate in skin and be biologically cleared through absorption. In one embodiment, the bioabsorbable/biodegradable material is polyvinyl alcohol. When the bioabsorbable/biodegradable polymer is polyvinyl alcohol, the weight average molecular weight (MW) of the polyvinyl alcohol can be over 10,000 amu.

The base of the microneedle arrays of the present invention can be made of any material known in the art onto which the microneedles can be attached and subsequently separated. In one embodiment, the base of the microneedle array can be made of the same bioabsorbable/biodegradable material as the microneedles. Typically, the base can be flexible to facilitate application to curved skin surfaces.

The active agents that can be delivered using the microneedle arrays and associated methods are wide ranging. In one embodiment, the active agent can be a vaccine. The vaccine may be in the form of a micro-organism, either attenuated or non-attenuated, toxoids, protein subunits, and or conjugates. In embodiment, the active agent can be a nucleic acid. Non-limiting examples of nucleic acids that can be delivered include modified and unmodified DNA or RNA including siRNA and plasmids. In one embodiment, the active agent delivered by the microneedles of the microneedle array is a self-delivering siRNA.

In addition to the above listed active agents, additional active agents may be co-delivered with these active agents. In one embodiment, the microneedles of the microneedle arrays can include a second active agent selected from the group consisting of an immune system stimulator, a local anesthetic, a systemic drug, a hormone, and/or combinations thereof. In one embodiment, the immune system stimulator can be an interferon.

The microneedles of the microneedle arrays can also include an indicator capable of providing visual verification of microneedle placement in the skin surface. The indicator can be incorporated into the body of the microneedle, loaded onto the exterior of the microneedle, or loaded into the microneedle along with the drug, or any combination thereof. The indicator can, but need not be present in all of the microneedles of the microneedle array. Further, the indicator can be present in the microneedles of the microneedle array so as to form an image when the microneedles are embedded in the skin. In one embodiment, the image of the microneedle array can be specific to the particular active agent delivered by the microneedle array, thereby allowing a practitioner to know not only if the microneedles had been properly embedded into the skin of the subject, but also what type of active agent was received by the subject. This can be particularly advantageous when the subject may be in need of receiving multiple active agents, such as multiple vaccines. Furthermore, the image can be that of a creative or fun nature for the enjoyment and purposes of children or others receiving vaccination as such visual indicators will remain as a “tattoo” on the skin until the microneedles dissolve or are absorbed.

Generally, any biologically acceptable indicator known in the art can be used. In one embodiment, the indicator can be a dye. In one aspect, the dye can be deposited onto the exterior of at least one of the microneedles on the microneedle array using an ink-jet apparatus, by contacting the needles with a surface upon which the dye is previously deposited in a pattern, or by contacting dye with needle structures individually in an indexed fashion. In another embodiment, the indicator can be one that is only visible upon direct application of a light source. In one aspect, the indicator can be visible only after application ultra-violet and/or infrared light. The indicator can also be a biological indicator. In one embodiment, the indicator can be a fluorophore such as fluoresceine. In another embodiment, the indicator can be a bioluminescent enzyme such as a luciferase.

Generally, the needles may be loaded with an active agent such as those described above either by including this agent in the bulk material from which the needle structures are formed, by adding material to the surface of the film from which the needles are pulled, or by loading the needles after they are fully formed, or by a combination of these methods.

In the case in which the bulk material is loaded, an active agent payload, such as a fluorescein model drug may be co-dissolved with the polymer or dry component(s) of the film, or it may be dissolved (or suspended if poorly soluble) in this viscous solution prior to dispensing as a film from which needle structures will be pulled. An active agent included in this manner will be distributed throughout the needle structures and the backing material in rough proportion to the quantity of material in each part.

In the case in which payload material is added to the surface of the film, without wishing to be bound to a particular interpretation, it is observed that material on the surface of the film from which needle structures are formed is preferentially drawn into the needle structures pulled from this film, and needles thus formed are subsequently found to be enriched in any material that was thus surface-loaded relative to the quantity of that material in an equivalent volume of the backing material. Thus surface-loading of the film provides an enhancement of the efficiency of use of an active agent. For example, an ethanolic solution of 1% wt fluorescein model drug can be applied to the surface of a PVA film at a loading of about 20 uL per square cm, prior to needle formation. The resulting needles pulled from this surface-loaded PVA film are strongly colored by the fluorescein payload, while the backing material remains relatively pale in coloration, indicating that the majority of the fluorescein is incorporated into the needles. Similarly a second, thinner PVA film containing a drug can be applied in the same manner, or a film containing material to be incorporated besides PVA, with similar preferential incorporation into the needle structures.

In the case in which payload material is loaded into/onto the needle structures after these structures are fully formed, the needles are brought into brief contact with a solution containing active agent dissolved or suspended in a volatile phase, and subsequently this phase is evaporated, depositing the active payload as a component of the needle structure. In the case that the volatile phase will also swell or dissolve the material of which the needle is formed, for example a water solution in contact with a PVA needle, it is understood that the needle can additionally imbibe the solvent material, and can carry payload material into the matrix of structural material that forms the needle. For example, a 1% fluorescein solution in water will efficiently wet the surface of a dry PVA needle structure, being drawn into superficial channels along the needle length. Because PVA is hydrated and solubilized by water, a needle thus exposed becomes flexible and pliant, indicating that the water has diffused into the bulk PVA material comprising the needle structure. Coloration of a needle thus treated indicates that the fluorescein payload material also penetrates into the needle structure. Additionally, though such loading contact time is brief, typically less than one second, it is understood that some dissolution and re-deposition of the PVA needle material must take place, which may further incorporate payload into the bulk of the needle structure.

As discussed above, the present invention also provides for methods of vaccinating a subject as well as a method for minimizing the amount of vaccine required to effectively vaccinate a subject against a disease for which the vaccine is effective is provided. In one embodiment, a method of vaccinating can include provoking an immune response in a subject in need of vaccination and delivering a vaccine concomitantly with the provocation of the immune response. It is noted that the provoking of the immune response is intended to refer to an immune response that is in addition to the immune response that would be associated with the delivery of the vaccine. In some aspects, the immune response may be a localized response, at or around, the site of the microneedle administration. By provoking the immune response, the vaccine can have an increased effectiveness in achieving the desired inoculation. In one aspect of the invention, the provocation of the immune response can be accomplished, at least in part, by leaving the microneedles in the skin until they are absorbed by the subject's body. Without being limited by theory, it is believed that the microneedles persistence in the skin tissue may stimulate an increased immune response, including but not limited to in the skin, thereby enhancing the ability of the immune system to recognize the presence of the vaccine, and thus elevate the immune response to a higher level than the vaccine alone. This enhanced immune response can function to increase the effectiveness of the vaccine. In particular, in one embodiment of the invention, the presence of the microneedles enhances the immune response such that the effective amount of vaccine sufficient to provide inoculation to the subject is less than the amount of vaccine needed when delivered via intramuscular injection. Accordingly, in another embodiment, a method of minimizing the amount of vaccine required to effectively vaccinate a subject against a disease for which the vaccine is effective is provided. The method includes stimulating an immune response in the subject at a vaccination site concomitantly with delivery of the vaccination. Because of the body's increased ability to recognize the vaccine due to the immune response concomitantly sparked, less vaccine is required than would otherwise be required with other vaccination methods, for example, intramuscular injection.

### EXAMPLES

### Example 1

### Microneedle Array Device Preparation

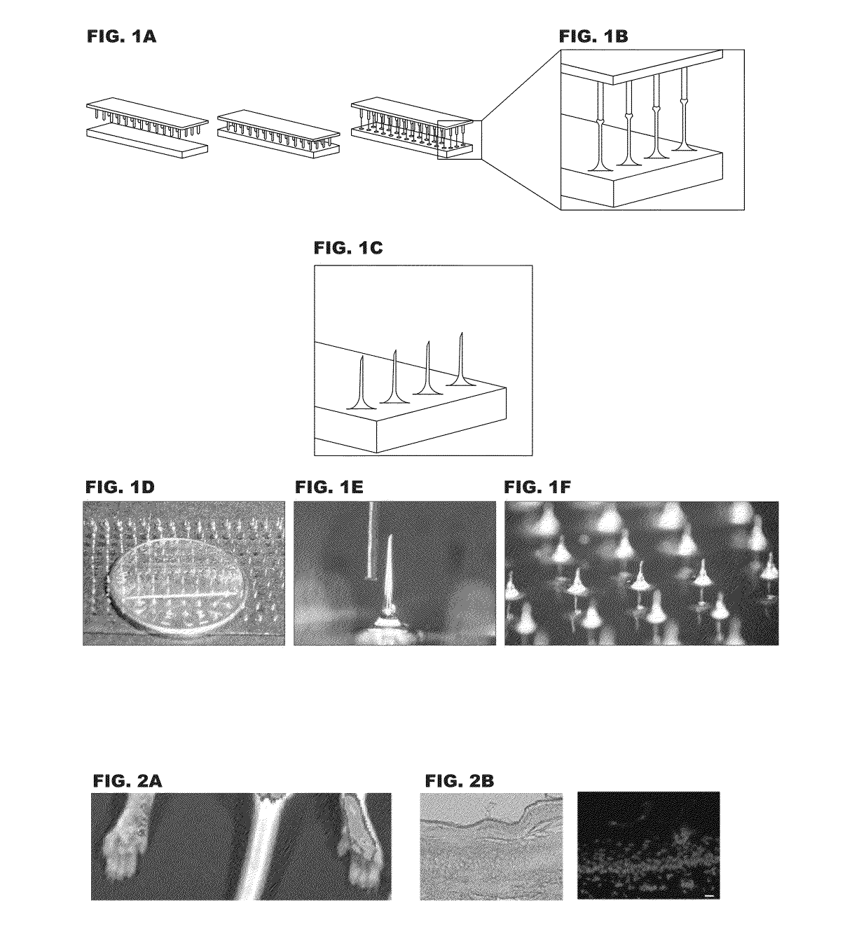

Microneedle arrays were produced by bringing a pin template into contact with a 1 mm thick film of 20% wt. polyvinyl alcohol (PVA) solution (FIG. 1A, left and middle panels), and withdrawing the template under a controlled air flow to produce fiber-like structures with loadable channels (FIG. 1A, right panel and B). The dried needle structures are separated from the template, and mechanically trimmed to a uniform height with sharp beveled tips (FIG. 1C). Typical fabrication and drying of microneedle arrays occurs at or below 50° C. (as low as 30° C., data not shown), below established siRNA degradation temperatures. The device is removed from the substrate as a regular array of dissolvable microneedles with an integral material backing (FIG. 1D). Typically, the microneedles on the microneedle arrays can be less than 100 μm thick, with a sharp tip only microns across (FIG. 1E). Microneedle arrays can be loaded with aqueous solutions or suspensions of payload materials, such as nucleic acids, drugs, vaccines, or other therapeutic agents. Multiple payloads can be delivered from separate needle populations on the same array (FIG. 1F). To show the flexibility of loading, phycobiliprotein R-phycoerythrin and fluorescein were alternatively loaded on arrays, demonstrating that multiple cargos can be delivered with a single device (FIG. 1F).

### Example 2

### Microneedle Array Device Preparation, Delivery and Application

Microneedle arrays were produced using an assembly of commercially available “pin headers” (Header Strip 80 pin dual row 1 mm spacing, PED-80S-P2, PAN PACIFIC, Santa Cruz, Calif.), as templates. The fabrication of the microneedles was accomplished by bringing the template pattern of projections into contact with a 1 mm thick film of 20% wt. polyvinyl alcohol (SPECTRUM, Gardena, Calif.) polymer solution on a glass substrate (Microscope Slides #1324L Globe Scientific, Paramus, N.J.). Following contact with the polymer film, the projections were withdrawn a distance of 1 cm over 13 s under a uniform airflow of 3.0 m/s at 45° C. Under these conditions, needle structures can be formed in the film with a hollow interior or exterior groove, which can be subsequently loaded through “capillary action.” Such microneedles were produced and mechanically trimmed to a nominal 1.0 mm length and 45 degree bevel, and loaded with “cargos” including nucleic acids (siRNAs and plasmids, see below), proteins (phycobiliprotein R-phycoerythrin) or dyes (e.g. fluorescein). Subsequent to loading, the microneedle arrays were incubated in a 50° C. vacuum oven evacuated to −18 cm mercury pressure for four hours to “harden” microneedles to facilitate skin penetration (the thin backing layer remains flexible, allowing conformation to skin contours during application). Microneedle arrays were applied manually to subjects such that the needle tips pierced the outermost skin surface by giving to the back of the array a single flick with the finger and were left in place for 20 minutes. During this period, the needle tips hydrated below the skin surface and softened to form a viscous gel plug. After this hydration period, the dry outer portion was removed, leaving the hydrated portions, with their cargo, embedded in the application site.

### Example 3

### Analysis of siRNA Distribution in Mouse Footpad Skin

Microneedle arrays were loaded with ˜20 ng/microneedle of the DY-547 (a Cy3 analog; 557 nm excitation, 570 nm emission) fluorescently-tagged siRNA mimic siGLO Red or Accell Red Non-Targeting siRNA (Dharmacon Products, Thermo Fisher Scientific, Lafayette, Colo.). The microneedle arrays were applied to the left paw of an FVB mouse using four consecutive applications of arrays (1×4) containing four microneedles each. Each microneedle array was applied for 20 minutes to maximize hydration of the penetrating microneedles. As a positive control, the right paw received 0.5 μg of siGLO Red in 50 μl PBS solution via intradermal injection. Mouse paws were imaged in an IVIS 200 imaging system using the DsRed filter set (excitation at 460-490 nm and 500-550 nm; emissions at 575-650 nm) during 1 s acquisition time. The resulting emitted light was quantified using Livinglmage software (Caliper LifeSciences), written as an overlay on Igor image analysis software (WaveMetrics, Inc; Lake Oswego, Oreg.). DsRed background was subtracted and raw values were reported as photons per second per cm² per steradian. Microneedle arrays in 3×5 array format were also applied to FVB or Tg hMGFP/CBL mice. Following CO₂ asphyxiation at the indicated times, skin tissue was dissected and frozen sections analyzed using a red filter set (546 nm excitation; 580 nm emission) in an Axio Observer Inverted Fluorescence Microscope (Zeiss, Thornwood, N.Y.) to visualize release from microneedle and biodistribution of siRNAs in skin. The images were taken with an AxioCam MRm (Zeiss) camera using AxioVs40 V4.6.3.0 software (Zeiss).

### Example 4

### Microneedle Array Delivery of siRNA Across the Stratum Corneum

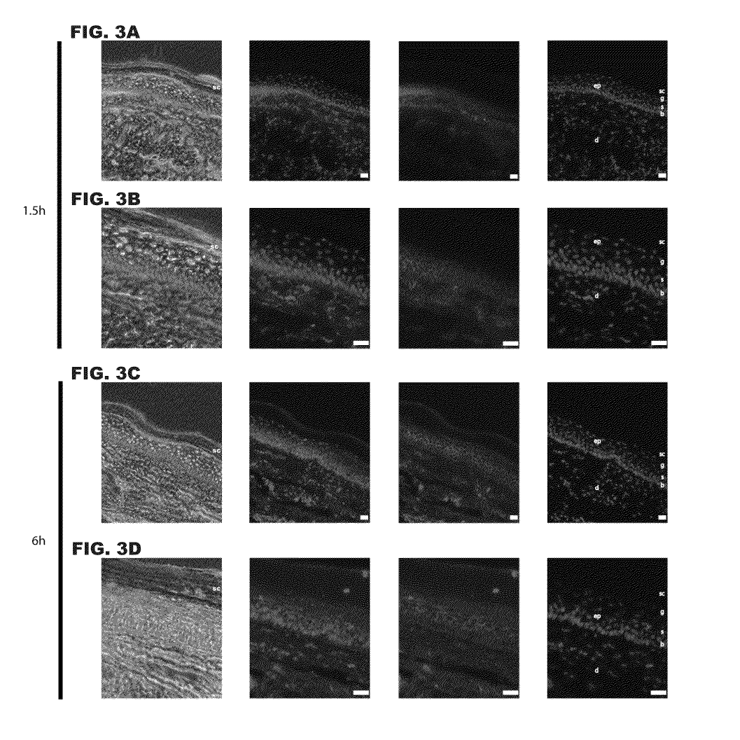

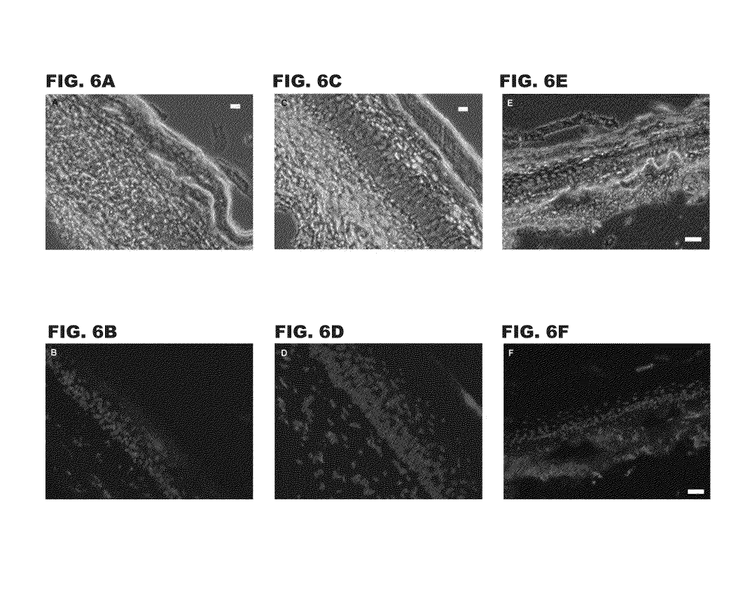

Microneedle arrays were loaded with a fluorescently-tagged siRNA mimic (siGLO Red) and applied to the left paw footpad. As a control, siGLO Red in PBS solution was injected intradermally into the right paw. Localized fluorescence corresponding to individual microneedle penetration sites could be visualized in vivo (FIG. 2A). After four applications of arrays (each containing four microneedles corresponding to a total deliverable dose of approximately 160-320 ng), the signal detected in the left paw was equal to the signal detected following intradermal injection of 0.5 μg into the right paw (data not shown). Individual microneedles loaded with siGLO Red could be observed penetrating the stratum corneum barrier to form localized depots in both the epidermis (FIG. 2B, and FIG. 6) and dermis (FIG. 6).

### Example 5

### Silencing of CBL/hMGFP Reporter Gene in Transgenic Mouse Epidermis

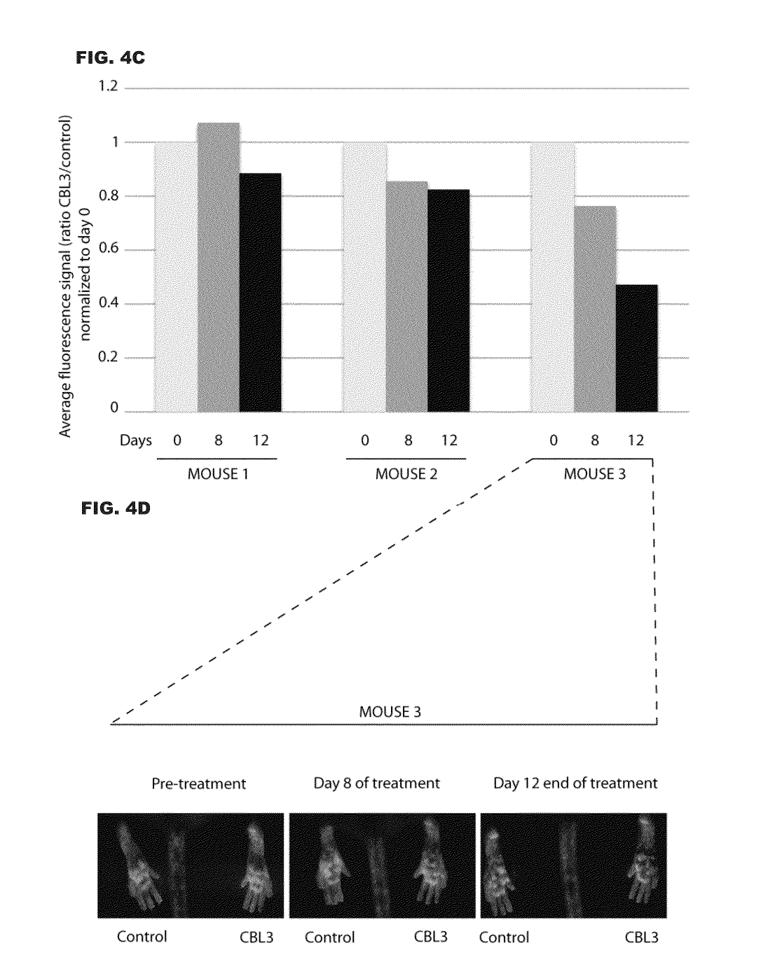

In order to test the ability of microneedle arrays to deliver functional CBL3 siRNA, mouse footpads were treated and the effect on reporter gene expression was analyzed. Three microneedle arrays (3×5 microneedle arrays) were applied to paw skin every other day for 12 days. On day 13′ the mice were sacrificed, palm skin excised and CBL/hMGFP reporter mRNA levels were measured by RT-qPCR (FIG. 4A). A significant reduction (up to 50%) in expression was found in all three CBL3 siRNA-treated palms compared to the counterpart palms treated with non-specific control siRNA. No significant reduction was found in footpads treated with non-specific siRNAs and footpads treated with empty microneedle arrays (data not shown), although variability was observed between individuals. In addition to mRNA reduction, decreased signal from reporter protein was also observed by fluorescence microscopy of footpad sections (FIG. 4B) of skin treated with CBL3 Accell siRNA (right panel) but not control siRNA (left panel). The reduced reporter mRNA levels on each mouse analyzed correlated well with the amount of protein reduction measured by in vivo fluorescence imaging (FIG. 4C). Representative images (mouse 3) demonstrating inhibition are shown in FIG. 4D.

### Example 6

### In vivo Delivery of siRNAs Using Microneedle Arrays and Analysis of Gene Silencing

Microneedle arrays containing approximately ˜20 ng/microneedle of Accell CBL3 and non-specific control Accell siRNAs (Dharmacon) were applied for 20 minutes to right and left footpads, respectively, of Tg CBL/hMGFP mice anesthetized with 2% isofluorane. Mice were treated every 48 h with three arrays (4×5) per treatment for 12 days. The mice were sacrificed on day 13 of the experiment and treated footpad tissues removed. Footpad skin from one mouse per cohort was embedded in O.C.T. compound (Tissue-Tek®, Torrance, Calif.) and frozen in dry ice. Vertical cross sections (10 μm) were prepared and mounted with Hydromount™ (National Diagnostic, Highland Park, N.J.) containing 1 μg/ml DAPI (Sigma) for nuclear staining Tissue sections were imaged with a GFP filter set (470 nm excitation, 525 nm emission) in an Axio Observer Inverted Fluorescence Microscope equipped with an AxioCam MRm camera to visualize transgene fluorescence as described above. Prior to the initial treatment, isoflurane-anesthetized (2%) mice were intravitally-imaged using the Maestro Optical imaging system (CRi Inc., Woburn, Mass., USA) as previously described and again at days 8 and 12 of the treatment regimen. Images were taken with an excitation filter of 445-490 nm and a long-pass emission filter (515 nm). Images were automatically captured at 10 nm windows from 500 to 700 nm using the Maestro software (exposure times were automatically calculated). Spectral un-mixing of the resulting cube image was performed using a user-defined hMGFP protocol. Each spectrum was set manually by un-mixing auto fluorescence from a negative non-hMGFP expressing mouse analyzed in parallel with a Tg CBL/hMGFP positive mouse. Standardized conditions and subject positioning for image acquisition facilitated meaningful comparison of data collected on different days. Regions of interest were drawn in the palm of each paw and the average signal (counts/s/mm²) was calculated. The ratio of average signal in right (CBL3) versus left (non-specific control) paws was calculated for each mouse and normalized with respect to the pretreatment analysis data. The un-mixed signal was pseudo-colored green (FIG. 4D).

For RT-qPCR analysis, three mice were sacrificed and skin tissues removed, pad and palm regions separated from the footpad, and frozen directly in dry ice. Tissue was homogenized in a “bead beater” instrument (FastPrep®-24, FP24, from MP Biomedicals, Solon, Ohio) and RNA isolated as previously described. RNA was reverse transcribed using the Superscript III First Strand Synthesis system (Invitrogen) and qPCR was run in the ABI 7500 Fast Sequence Detection system (Applied Biosystems, Foster City, Calif.) using standard procedures. hMGFP and mouse keratin 14 (Catalog #Mm00516876) Taqman® Gene Expression Assays were used as previously described. All data points reported are the mean of 3 replicate assays and error is reported as the standard error.

### Example 7

### Delivery of Luciferase Expression Plasmid to Mouse Skin and Visualization of Expressed Reporter Protein by Intravital Imaging

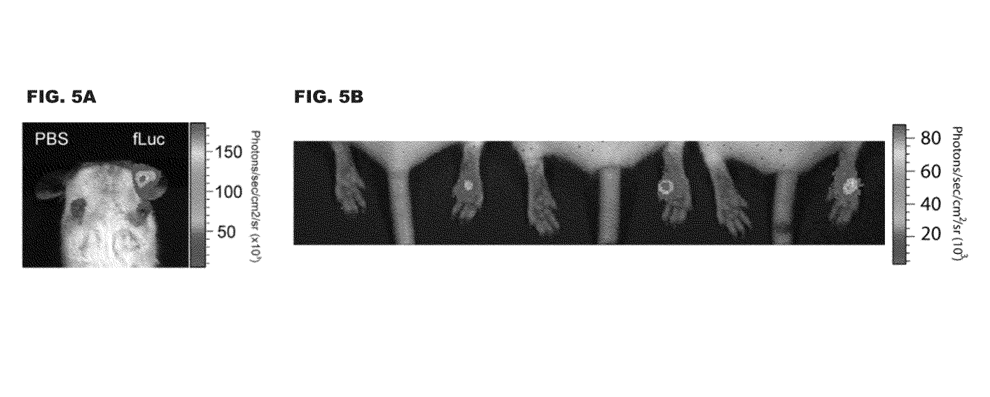

Microneedle arrays containing firefly luciferase (fLuc) expression plasmid were applied to mouse back, ear and paw skin. Following luciferin administration 24 h later, luciferase expression was assayed by in vivo bioluminescence imaging (BLI). Luminescence was detected in all the treated skin types (left ear and footpad are shown in FIGS. 5A and B, respectively; data not shown for back skin expression) while no expression was found in skin treated with microneedle arrays loaded with PBS vehicle alone. To test the consistency of plasmid delivery and expression in mouse paw skin, 12 microneedles (each containing a deliverable payload of approximately 12 ng per microneedle of pGL3-CMV-Luc) were applied per mouse paw on day 1 and again on day 2, and imaged on day 3 by in vivo bioluminescent imaging (FIG. 5B). We observed consistent reduction in luciferase expression using this method (FIG. 5B and data not shown; experiment was repeated five times with similar results), demonstrating that the microneedle arrays are able to reliably deliver functionally active plasmid DNA to skin cells.

### Example 8

### In vivo Delivery of Plasmid DNA Using Microneedle Arrays

Microneedle arrays (3×4) were loaded with approximately 12 ng/microneedle of pGL3-CMV-Luc plasmid and applied back, ear and footpad skin of anesthetized (2% isofluorane) mice for 20 minutes. 24 and 48 h following the array application, the mice were IP injected with luciferin (100 μl of 30 mg/ml luciferin; 150 mg/kg body weight) and the live anesthetized mice imaged 10 min later in the IVIS 200 Imaging System as previously described. The resulting light emission was quantified using Livinglmage software as described above.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

1. A method of providing visual verification of microneedle placement in a skin surface, comprising,
providing a microneedle array having a plurality of microneedles attached thereto, said microneedles including an indicator
applying the microneedle array to a skin surface of a subject such that the microneedles are embedded into the skin surface,
verifying successful application of the microneedles through observation of the indicator's presence in the skin.

2. The method of claim 1, wherein the indicator is a dye.

3. The method of claim 1, wherein the indicator forms an image on the skin of the subject.

4. The method of claim 1, wherein the indicator is visible when light is shown onto the microneedles.

5. The method of claim 1, wherein the indicator is a fluorophore.

6. The method of claim 5, wherein the flourophore includes fluorescein.

7. A system for delivering siRNA to a subject, comprising:
an amount of a self-delivering siRNA
a microneedle array, said microneedle array comprising
a base, and
a plurality of microneedles attached to the base, said microneedles comprising a bioabsorbable/biodegradable material and being and configured to be detached from the base after being embedded in a skin surface.

8. The system of claim 7, wherein the microneedles include a plurality of longitudinal channels.

9. The system of claim 8, wherein the self-delivering siRNA are present in the longitudinal channels.

10. The system of claim 7, wherein the bioabsorbable/biodegradable material is selected from the group consisting of polyvinyl alcohol, polyvinylpyrrolidone, chitin, carboxymethyl cellulose, methyl cellulose, ethyl cellulose, sodium alginate, carrageenan, carbomer, alginates, and other polysaccharides, shellac, zein, glucose, sucrose, maltose, trehalose, amylose, dextrose, fructose, mannose, galactose, fructose, and mixtures thereof.

11. The system of claim 7, wherein the bioabsorbable/biodegradable material includes polyvinyl alcohol.

12. The system of claim 7, wherein the microneedles include a lower portion and an upper portion, wherein the lower portion is adjacent to the base and the upper portion is opposite the base.

13. The system of claim 12, wherein the upper portion of the microneedle is compositionally distinct from the lower portion of the microneedle.

14. The system of claim 12, wherein the active agent is only present in the upper portion of the microneedle.

15. The system of claim 12, wherein the active agent is only present in the lower portion of the microneedle.

16. The system of claim 7, wherein at least some of the microneedles of the microneedle array include a second active agent.

17. The system of claim 16, wherein the second active agent is selected from the group consisting of: an immune system stimulator, anesthetic, a systemic drug, a hormone, and combinations thereof.

18. The system of claim 17, wherein the immune system stimulator is an interferon.

19. A method of vaccinating a subject in need thereof, comprising:
providing a microneedle array comprising a base and microneedles attached to the base, said microneedles being loaded with an amount of a vaccine sufficient to provide inoculation to the subject,
applying the microneedle array to a skin surface of a subject such that the microneedles are embedded into the skin surface,
separating the base of the microneedle array from the microneedles such that the microneedles remain embedded in the skin surface and the base is removed from the skin surface, and maintaining the microneedles in the skin surface until the microneedles are absorbed by the subject.

20. The method of claim 19, wherein the maintaining of the microneedles in the skin surface causes an enhanced immune response.

21. The method of claim 20, wherein the enhanced immune response increases the effectiveness of the vaccination.

22. The method of claim 21, wherein the amount of vaccine is sufficient to provide inoculation to the subject is less than the amount of vaccine needed when delivered via intramuscular injection.

23. A method of vaccinating, comprising:
provoking an immune response in a subject in need of a vaccination,
delivering a vaccine to the subject concomitantly with the provocation of the immune response.

24. A method of minimizing an amount of vaccine required to effectively vaccinate a subject against a disease for which the vaccine is effective, comprising: stimulating an immune response in the subject at a vaccination site concomitantly with delivery of a vaccine.

25. The method of claim 24, wherein the delivery of the vaccine is accomplished through the embedding and leaving of microneedles into a skin surface of a subject and the leaving of the microneedles in the skin provokes the immune response.

26. The method of claim 25, wherein the vaccine is delivered by the microneedles that are left in the skin.
